(19) United States
(12) Patent Application Publication
(10) Pub. No.: US 2015/0297134 A1
(43) Pub. Date: Oct. 22, 2015

(54) METHODS AND SYSTEMS FOR CARDIAC MONITORING WITH MOBILE DEVICES AND ACCESSORIES

(71) Applicant: AliveCor, Inc., San Francisco, CA (US)

(72) Inventors: David E. ALBERT, San Francisco, CA (US); Nupur SRIVASTAVA, San Francisco, CA (US); Nathaniel FOX, San Francisco, CA (US); Ravi GOPALAKRISHNAN, San Francisco, CA (US)

(21) Appl. No.: 14/692,563

(22) Filed: Apr. 21, 2015

Related U.S. Application Data
(60) Provisional application No. 61/982,002, filed on Apr. 21, 2014.

(51) Int. Cl. A61B 5/00; A61B 5/0416; A61B 5/0408; A61B 5/04; A61B 5/044

(52) U.S. Cl. CPC A61B 5/681; A61B 5/04012; A61B 5/044; A61B 5/04085; A61B 5/0006; A61B 5/486; A61B 5/0416; A61B 5/0024; A61B 2562/227; A61B 2562/0214

(57) ABSTRACT

Systems, devices, and methods are provided for measuring an electrocardiogram (ECG) or other cardiac parameters with an attachable monitoring device. The attachable monitoring device may be coupled to a wearable monitoring device or wearable monitoring devices. Sensor electrodes are built into the main body of the attachable monitoring device to sense a biometric parameter of a user.

### CROSS-REFERENCE

This application claims the benefit of U.S. Provisional Patent Application Ser. No. 61/982,002, filed on Apr. 21, 2014, which is incorporated herein by reference in its entirety.

### BACKGROUND

The present disclosure relates to systems, devices, and methods for managing health and disease with portable electronic devices. In particular, the present disclosure relates to systems, devices, and methods for managing cardiac health with mobile computing and/or telecommunications devices.

The use of smartphones, tablet computers, wearable computers, smartwatches and “smart” accessories is becoming increasingly prevalent. Smartphones are almost ubiquitous in high income countries and are increasingly popular in middle and low income countries as the costs of production decrease and Internet access becomes more available. This increased prevalence of computing power and devices offers many opportunities for improved ways of monitoring health and placing health management more in the control of the end-user or patient rather than medical professionals who may be relatively inaccessible.

Applications have been provided for smartphones, tablet computers, wearable monitoring devices, and the like to provide the user with the ability to track various parameters for health. The user may be able to enter into the application(s) his or her weight and diet, for example. An on-board accelerometer of the device may track the sleep and/or activity levels of the user. Heart rate may be monitored using the on-board camera and flash of the device and some devices even include on-board electrodes for monitoring heart rate. Accessories for these devices have been available to track activity levels and other health parameters as well.

Cardiovascular disease is a leading cause of death in the world and is prevalent in the populations of high-income and low-income countries alike. Heart rate measurement, blood pressure measurement, and electrocardiography are widely used techniques for diagnosing the cardiovascular health of patient. While device to measure heart rate and blood pressure are highly prevalent and accessible, many of these devices can be less than ideal in at least some cases. For example, such devices may provide only the heart rate and blood pressure of a patient but fail to provide many other important diagnostic parameters which can be determined or derived from heart rate and blood pressure. Also, these devices may not be portable and it may be difficult to make continuous measurements which can provide important health and diagnostic information. Electrocardiography may not be accessible to users as would be ideal as access to professional clinics is often still required.

### SUMMARY

Recognized herein is a need for improved health monitoring systems, devices, and methods to address one or more of these challenges and which may take advantage of the increased prevalence of computing power and telecommunications. This increased prevalence may offer opportunities for improved ways of monitoring health and placing health management more in the control of the end-user or patient rather than medical professionals who may be relatively inaccessible.

The present disclosure relates to systems, devices, and methods for managing health and disease with portable electronic devices. In particular, the present disclosure relates to systems, devices, and methods for managing cardiac health with mobile computing and/or telecommunications devices.

Aspects of the present disclosure provide systems, devices, and methods for health monitoring. Such systems, devices, and methods may take advantage of the increased prevalence of computing power and telecommunications and provide accessible ways for a user to monitor his or her health. Many smartphones, tablet computers, wearable monitoring devices, and the like available or soon to be available in the market may have the ability to interface with a wearable accessory component such as a “smartwatch.” The smartwatch or secondary computing device may include a display and a user interface through which the user can interact with the primary computing device. In some embodiments, the wearable component or device may be a wearable monitoring device or wearable monitoring device, terminal or display (e.g., in communication with data over a network, such as a cloud network, a cellular network or any other network). In some embodiments, the wearable component or device may provide at least a portion of functions provided by smartphones, tablet computers, wearable monitoring devices, and the like. Any aspects of the disclosure described in relation to secondary devices may equally apply to primary devices (e.g., wearable monitoring devices) at least in some configurations, and vice versa.

In many aspects, systems, devices, and methods are provided such that the smartwatch or secondary computing device can provide additional health monitoring features. For example, heart rate (HR) and/or an electrocardiogram (ECG) may be taken with such smartwatch or secondary computing device associated devices. The straps of a smartwatch may be interchangeable and embodiments of the present disclosure may provide a smartwatch strap having a health monitoring function. The strap may comprise one or more electrodes configured to contact the skin of the user to continuously or on contact measure the heart rate or electrocardiogram of a user. The strap may also comprise an output to communicate the measured parameter to the smartwatch or primary computing device (e.g., the smartphone, the tablet computer, etc.) The output may comprise a WiFi transmitter, a Bluetooth transmitter, an audio or ultrasound acoustic transmitter or speaker (e.g., as described in U.S. Pat. Nos. 8,301,232 and 8,509,882, U.S. patent applications Ser. Nos. 13/108,738, 13/752,048, 13/964,490, 13/969,446 and 61/872,555), or the like.

The smartwatch or secondary computing device may also be used to alert the user to take one or more health measurements in timed intervals or when the primary computing device determines that it is appropriate to make such measurements. For example, a set of instructions may be provided on a memory or a machine-readable non-transient storage medium of the primary or secondary computing device so that the smartwatch or secondary computing device when executing the set of instructions learns when taking an ECG or other health monitoring is appropriate. Such learning may be based on environmental cues or user input for example through an activity sensor, a timer or clock, a location of the user, the state of the device (e.g., charging, unplugged, screen on/off, WiFi on/off, Bluetooth on/off, audio output on/off, etc.), sweat (e.g., through a moisture sensor), or other received metrics from third devices linked to the primary device (e.g., a blood pressure monitor.) Such intelligent prompting for user health monitoring may also be applicable for standalone computing devices without a secondary computing accessory such as a smartphone. For example, a smartphone or a tablet computer of the user may be configured to alert the user (e.g., through audio, visual display, vibration, etc.) to monitor his or her health parameter(s) based on learned or environmental cues.

In many aspects, systems, devices, and methods are provided for accessibly and conveniently measuring cardiac parameters such as heart rate (HR), heart rate variability (HRV), or R-R interval variability. A heart rate monitor may be incorporated into a secondary computing device worn by the user such as a smartwatch. The secondary computing device may provide continuous HR measurements through which HRV can be determined. Embodiments may also provide methods through which HRV can be determined using other accessory devices which can measure HR. For example, while the on-board camera and flash of a portable computing device or the on-board electrodes may be used to measure heart rate, the methods of the present disclosure may determine HRV based on the measured heart rate. Improved methods of determining HR and HRV using on-board hardware of a computing device may also be provided. For example, the camera of the computing device may be used to observe the user for variation in skin flushing of a user is to determine HR and/or HRV.

Described herein is a method for monitoring a biometric parameter of a user. The method comprises receiving, by the user, an attachable monitoring device comprising a processor and a sensor, wherein the attachable monitoring device is configured to couple with a wearable monitoring device of the user. The method further comprises coupling, by the user, the attachable monitoring device with the wearable monitoring device sensing, with the sensor, a biometric parameter of the user. The method further comprises transmitting the biometric parameter of the user to the wearable monitoring device of the user.

In an embodiment, the coupling step comprises both a physical and functional coupling of the attachable monitoring device with the wearable monitoring device. In an embodiment, the physical coupling comprises a snap-on coupling. In an embodiment, the functional coupling comprises a hardwire electronic coupling between the attachable monitoring device and the wearable monitoring device. In an embodiment, the attachable monitoring device further comprises a wireless transmitter, and the functional coupling comprises a wireless coupling between the attachable monitoring device and the wearable monitoring device. In an embodiment, the attachable monitoring device comprises a watch band, and the wearable monitoring device comprises a smartwatch. In an embodiment, the biometric parameter comprises an electrocardiogram. In an embodiment, the processor is configured to activate said sensor. In an embodiment, the processor is configured to analyze said sensed biometric parameter. In an embodiment, the method further comprises displaying the biometric parameter on a display of said wearable monitoring device.

Also described herein is an attachable monitoring device comprising a body having a first surface and a second surface. The body is configured to removably couple with a wearable monitoring device, and the body comprises a first sensor positioned on the first surface, and the first sensor is configured to sense a biometric parameter of a user. The body comprises a processor coupled to the first and second sensors, and a coupler configured to couple said body with said wearable monitoring device.

In an embodiment the coupler both physically and functionally couples the body with the wearable monitoring device. In an embodiment, the physical coupling comprises a snap-on coupling. In an embodiment, the functional coupling comprises a hardwire electronic coupling between said attachable monitoring device and said wearable monitoring device. In an embodiment, the body further comprises a wireless transmitter, and said functional coupling comprises a wireless coupling between said attachable monitoring device and said wearable monitoring device. In an embodiment, first sensor is configured to measure an electric potential on a first skin surface of a user, and wherein said body further comprises a second sensor positioned on said second surface, wherein said second sensor is configured to measure an electric potential on a second skin surface of said user. In an embodiment, the attachable monitoring device further comprises a non-transitory computer-readable storage media encoded with a computer program including instructions executable by said processor to cause said processor to generate an electrocardiogram comprising said electric potential on said first skin surface of said user. In an embodiment, the computer program further causes said processor to transmit said electrocardiogram to said wearable monitoring device. In an embodiment, the computer program further causes said processor to instruct said user to contact said first and said second sensors. In an embodiment, the wearable monitoring device comprises a smartwatch and said body comprises a watchband.

Described herein is a cardiac parameter monitoring method. The method comprises the step of determining, with a mobile computing device, a need to record an electrocardiogram, wherein the need to record the electrocardiogram is based on a measured biometric parameter of a user. The method describes transmitting, to a watchband configured to couple with a smartwatch, an executable command to record an electrocardiogram with the watchband, wherein the watchband comprises a receiver, a processor coupled to the receiver, and two electrodes coupled to the processor. The method further comprises receiving, with the receiver, a signal from the mobile computing device comprising the executable command to record an electrocardiogram with the watchband. The method further comprises receiving, with the processor from the receiver, the signal from the mobile computing device comprising the executable command to record an electrocardiogram with the watchband. The method further comprises recording an electrocardiogram with the two electrodes in response to the executable command to record an electrocardiogram received from the processor.

In an embodiment, watchband further comprises a transmitter coupled to the processor. In an embodiment, the method further comprises transmitting, with the transmitter, a signal comprising the electrocardiogram to a mobile computing device. In an embodiment, the method further comprises the step of causing the smartwatch to display the electrocardiogram on a face of the smartwatch. In an embodiment, the watchband reversibly couples to the smartwatch. In an embodiment, the method further comprises instructing a user to contact the watchband with a skin surface. In an embodiment, one of the two electrodes is positioned on a first surface of the watchband and one of the two electrodes is positioned on a second surface of the watchband. In an embodiment, the measured biometric parameter comprises a heart rate. In an embodiment, the measured biometric parameter comprises a heart rate variability. In an embodiment, the measured biometric parameter comprises a blood pressure.

Also described herein is a watchband comprising a body having a first surface and a second surface, wherein the body is configured to removably couple with a smartwatch. The body comprises a first sensor positioned on the first surface, wherein the first sensor is configured to measure an electric potential on a first skin surface of a user, and a second sensor positioned on the second surface, wherein the second sensor is configured to measure an electric potential on a second skin surface of the user. The body comprises a receiver coupled to the first and the second sensors, wherein the receiver is configured to receive a first wireless signal. The body comprises a processor coupled to the receiver, and the first and second sensors. The body comprises a non-transitory computer-readable storage media encoded with a computer program including instructions executable by the processor to cause the processor to cause, in response to the first wireless signal received by the receiver, the first sensor to sense the electric potential on the first skin surface of the user and the second sensor to sense the electric potential on the second skin surface of the user.

In an embodiment, the watchband further comprises a transmitter coupled to the processor. In an embodiment, the computer program further causes the processor to transmit, with the transmitter, a second wireless signal comprising the electrocardiogram to a mobile computing device. In an embodiment, the computer program further causes the processor to cause the smartwatch to display the electrocardiogram on a face of the smartwatch. In an embodiment, the first wireless signal is transmitted from a computing device in response to a biometric parameter of the user. In an embodiment, the biometric parameter comprises a heart rate. In an embodiment, the measured biometric parameter comprises a heart rate. In an embodiment, the measured biometric parameter comprises a blood pressure. In an embodiment, the computing device comprises a smartphone. In an embodiment, the computer program further causes the processor to cause the smartwatch to display a message to the user to contact the first and the second sensors.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

The contents of U.S. patent applications having Ser. Nos. 14/328,962 (filed Jul. 11, 2014), 13/969,446 (filed Aug. 16, 2013), 13/108,738 (filed May 16, 2011), 12/796,188 (filed Jun. 8, 2010) along with the now expired U.S. Provisional Patent Applications having Ser. Nos. 61/872,555 (filed Aug. 30, 2013), 61/725,422 (filed Nob. 12, 2012) are incorporated herein by reference. Further, all publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1L show an embodiment of an attachable monitoring device comprising a cover that fits over a wristwatch as described herein.

FIG. 2A-FIG. 2K show an attachable monitoring device comprising a watchband comprising of straps as described herein.

FIG. 3A-FIG. 3H show anterior views of an embodiment of an attachable monitoring device comprising an essentially round jewelry such as, for example, a ring, bracelet, or earing.

FIG. 4A-FIG. 4F show anterior and lateral views of an attachable monitoring device comprising a necklace with a pendant.

FIG. 5 shows a schematic representation of an embodiment of an attachable monitoring device as described herein.

FIG. 6 shows a schematic of a method of measuring an electrocardiogram as described herein.

FIG. 7 shows a schematic of a method of notifying a user and measuring an ECG of the user as described herein.

FIG. 8 shows a schematic representation of a method for monitoring a user as described herein.

### DETAILED DESCRIPTION

Described herein are devices, systems, and methods for monitoring the heart health of a user. Before explaining at least one embodiment of the inventive concepts disclosed herein in detail, it is to be understood that the inventive concepts are not limited in their application to the details of construction, experiments, exemplary data, and/or the arrangement of the components set forth in the following description, or illustrated in the drawings. The presently disclosed and claimed inventive concepts are capable of other embodiments or of being practiced or carried out in various ways. Also, it is to be understood that the phraseology and terminology employed herein is for purpose of description only and should not be regarded as limiting in any way.

In the following detailed description of embodiments of the described subject matter, numerous specific details are set forth in order to provide a more thorough understanding of the inventive concepts. However, it will be apparent to one of ordinary skill in the art that the inventive concepts within the disclosure may be practiced without these specific details. In other instances, well-known features have not been described in detail to avoid unnecessarily complicating the instant disclosure.

Further, unless expressly stated to the contrary, “or” refers to an inclusive or and not an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

Finally, as used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Described herein are devices, systems, and methods for monitoring the heart health of a user using a wearable device. In an embodiment, an attachable monitoring device couples with wearable monitoring device which comprises any item configured to be worn by a user and monitor any biometric parameter of said user. A wearable monitoring device may comprise a wearable computer such as, for example, a smartwatch or smartglasses. A wearable monitor may comprise a wearable sensor. In an embodiment, a wearable monitoring device is configured to be any item worn by a user. For example, an item worn by a user may comprise, for example, jewelry, or clothing. Jewelry may, for example, comprise a necklace, a bracelet, a watch, or a ring. Clothing, may comprise, a shirt, pants, shoes, a hat, an undergarment, eyewear, or shoes. A wearable monitoring device may comprise, for example, a smartwatch or smartglasses. The sensor devices or sensor accessories provided herein may be wearable by the user. Exemplary devices or accessories may be provided in the form(s) of a ring, a watch (e.g., a smartwatch), a strap, a button, a patch, a band (e.g., a chest band), a tattoo, a paste, etc. The wearable sensor devices may be removable from the user (e.g., mechanically fastened, adhesively attached, smeared on as a cream, etc.). Alternatively, the wearable sensor devices may be permanently attached to the user (e.g., as an implant or a tattoo). Exemplary sensor devices or accessories may comprise components that snap onto another user device or object, such as, for example, a band or a clip that snaps or clips onto a watch, a fitness or exercise device (e.g., a treadmill, an elliptical machine, a wheel frame of a bicycle or motorcycle, a steering wheel), or an everyday device (e.g., any device or object that a user may touch on a daily basis, such as a book, a magazine, a mug, a computer mouse, a keyboard, a chair, a bag, etc.). Thus, the sensor devices or accessories may include any form factor. Furthermore, the sensor devices or accessories may include wires (e.g., wires to connect to a primary computing device for data transmission) or a wireless transmitter (e.g., for data transmission). In some embodiments, the sensor devices or accessories may be touched by the user when they are mobile or “on the go” (e.g., while the user is driving and contact his or hands with a steering wheel, while the user is eating or drinking and his or hands are contacting a mug or other utensil, while the user is reading and his or hands are contacting a book or magazine cover, etc.).

The systems, devices, and methods of the disclosure may be used to enable episodic or continuous monitoring of user(s). The systems, devices, and methods herein may be advantageously used to enable home monitoring. In some embodiments, the monitoring may be associated with a given health condition of a user or patient (e.g., high blood pressure, obesity, age, etc.). In some embodiments, the monitoring may be performed on individuals without a known or given health condition (e.g., as part of a workout or training regimen).

Aspects of the present disclosure provide many ways of incorporating physiological sensing functions, particularly cardiac parameter sensing functions, into wearable monitoring devices.

In an embodiment, an attachable monitoring device may couple to an item worn by a user through a mechanism, for example, comprising a snap-on, an adhesive, inter-locking, or magnetic mechanism. For example, a wearable monitoring device may comprise a snap-on clip configured to fit around either a smartwatch housing or smartwatch band. A snap-on clip may be adjustable to snap-on to smartwatches having bodies and bands of different sizes. A snap on mechanism may comprise components configured to interlock when coupled, wherein a first interlocking component is positioned on the attachable monitoring device and a second interlocking component is positioned on a wearable monitoring device. An adhesive may comprise glue or a fabric such as, for example, Velcro. An interlocking mechanism may comprise, for example, a pin attached to the device that fits into a hole on a smartwatch housing or smartwatch band, or alternatively the pin may be on the smartwatch housing or band and the hole into which the pin fits may be on the device. A magnetic mechanism may comprise, for example, a magnet on the wearable monitoring device may magnetically couple with a smartwatch housing or band.

In an embodiment, an attachable monitoring device may be configured to removably couple to one or more wearable monitoring devices of the same kind or alternatively or additionally, a wearable monitoring device may be configured to couple with different types of wearable monitoring devices. For example, in an embodiment, an attachable monitoring device may couple with different smartwatches having, for example, different shapes or sizes. For example, in an embodiment, an attachable monitoring device may couple with a smartwatch as well as a necklace.

In an embodiment, an attachable monitoring device may be configured to functionally couple with a wearable monitoring device. For example, in an embodiment, an attachable monitoring device is configured to engage a port or plug on a wearable monitoring device to form a hardwire connection wherein electric signals may be transmitted directly from the attachable monitoring device to the wearable monitoring device through the port or plug of the wearable monitoring device. In an embodiment, a wearable monitoring device comprises a cradle or dock to receive the attachable monitoring device, wherein the cradle or dock comprises a port or plug that is configured to hard connect with the attachable monitoring device. In an embodiment, an attachable monitoring device comprises a transmitter that is configured to transmit a wireless signal to a wearable monitoring device.

In an embodiment, an attachable monitoring device comprises one or more sensors. A sensor may, for example, measure a biometric parameter of a user. A biometric parameter of a user may, for example, comprise a heart rate, a photoplethysmogram, a blood pressure, an electrocardiogram, a heart rate variability, a heart rate turbulence, or any other biometric parameter. In an embodiment, one or more sensors comprise electrodes configured to measure an electric potential on a skin surface of a subject. One or more sensors may comprise, for example, 8 sensors. One or more sensors may comprise, for example, 7 sensors. One or more sensors may comprise, for example, 6 sensors. One or more sensors may comprise, for example 5, sensors. One or more sensors may comprise, for example, 4 sensors. One or more sensors may comprise, for example, 3 sensors. One or more sensors may comprise, for example, 2 sensors. One or more sensors may comprise, for example, 1 sensor. In an embodiment, an attachable monitoring device comprises one or more sensors that record the same biometric parameter of a user. In an embodiment, an attachable monitoring device comprises one or more sensor that record difference biometric parameters.

In an embodiment, an attachable monitoring device comprises a transmitter. A transmitter may comprise a wireless transmitter configured to transmit a wireless signal. A wireless signal may, for example, comprise a Bluetooth signal, an RF signal, an Ultrasound signal, or a WiFi signal, or any other wireless signal. In an embodiment, a transmitter is configured to transmit a wireless signal comprising an electric potential measured on a skin surface of a user. In an embodiment, a wireless signal may comprise an electrocardiogram signal which comprises a difference in an electric potential on at least two different skin surfaces of a user.

In an embodiment, an attachable monitoring device comprises a battery to power some or all of the components of the attachable monitoring device. A battery may be rechargeable or replaceable.

In an embodiment, an attachable monitoring device comprises a receiver. A receiver may comprise a wireless receiver configured to receive a wireless signal. A wireless signal may, for example, comprise a Bluetooth signal, an RF signal, an Ultrasound signal, or a WiFi signal, or any other wireless signal. In an embodiment, a receiver is configured to receive a wireless signal comprising an executable command.

In an embodiment, an attachable monitoring device further comprises a processor. A processor is functionally coupled to one or more of one or more sensors, a transmitter, a battery, or a receiver. In an embodiment, a wireless signal received by said receiver comprises an executable command that is transmitted by the receiver to the processer. The processor is configured to execute an executable wireless signal received by the receiver. The processor may be configured to decode an encoded signal in an executable command. For example, in an embodiment, an executable command received by the processor may cause the processor to activate one or more sensors coupled to the processor. For example, an executable command received by the processor may cause the processor to cause a transmitter coupled to the processor to transmit a wireless signal. For example, an executable command received by the processor may cause the processor to cause one or more electrodes coupled to the processor to sense an electric potential on a skin surface of a user. For example, an executable command received by the processor may cause the processor to cause two or more electrodes coupled to the processor to sense an electrocardiogram of a user. For example, an executable command received by the processor may cause the processor to cause a transmitter coupled to said processor to transmit a wireless signal comprising of an electrocardiogram of a user.

Described herein is an attachable monitoring device configured to couple with smartwatch. In an embodiment, the attachable monitoring device comprises a cover which may, for example, comprise a skin, sleeve, wrap, or bag that fits over or around a smartwatch or traditional wristwatch. In an embodiment, the attachable monitoring device is configured to couple with a smartwatch housing or smartwatch band.

In an embodiment, an attachable monitoring device comprises a watchband. An attachable monitoring device comprising a watchband may couple with a traditional watch or a smartwatch. In an embodiment, an attachable monitoring device comprising a watchband couples with a smartwatch housing. In an embodiment, a an attachable monitoring device comprising a watchband further comprises a body comprising of either a single band or two straps and a buckle. The body may further comprise one or more of one or more sensors, a transmitter, a receiver, a battery, or a processor. The attachable monitoring device may comprise a smartwatch such as the attachable monitoring device. The main body of the attachable monitoring device may have two or more sensing electrodes placed thereon. For example, a first electrode may be positioned over the front periphery of the main body (e.g., over the dial or on/over the bezel) of the attachable monitoring device and a second electrode may be positioned over the back plate of the attachable monitoring device. The first electrode may be contacted with a right or left hand or fingers while the second electrode contacts the opposite wrist to measure heart rate, an electrocardiogram, body fat percentage, hydration, etc. The electrode(s) may be built into the main body of the attachable monitoring device and/or may be provided as a snap-on shell either replacing or overlaying the outer protective shell of the main body. In embodiments where the electrodes(s) are built into the main body of the attachable monitoring device, the sensor electrode(s) may be coupled to the processor of the attachable monitoring device through an internal wired connection. In embodiments where the electrode(s) are provided on a snap-on shell or case, the sensor electrode(s) may be coupled to the processor of the attachable monitoring device through a wired (e.g., Lightning, USB, FireWire, or the like) or a wireless connection (e.g., WiFi, cellular, Bluetooth classic, Bluetooth low energy, NFC, ultrasound data communication (as described, for example, in U.S. Pat. Nos. 8,301,232 and 8,509,882 and U.S. patent applications Ser. Nos. 13/108,738, 13/752,048, 13/964,490, 13/969,446, and 61/872,55), or the like) through the wireless transmitter and/or speaker and microphone of the attachable monitoring device (i.e., the snap-on shell or case may be provided with a wireless transmitter.)

In an embodiment, a user may touch the attachable monitoring device to contact a first side of the body with a first electrode and a second side of the body with a second electrode. Once properly contacted, the electrodes may measure the cardiac parameter of the user, e.g., a heart rate, an electrocardiogram (ECG), etc.

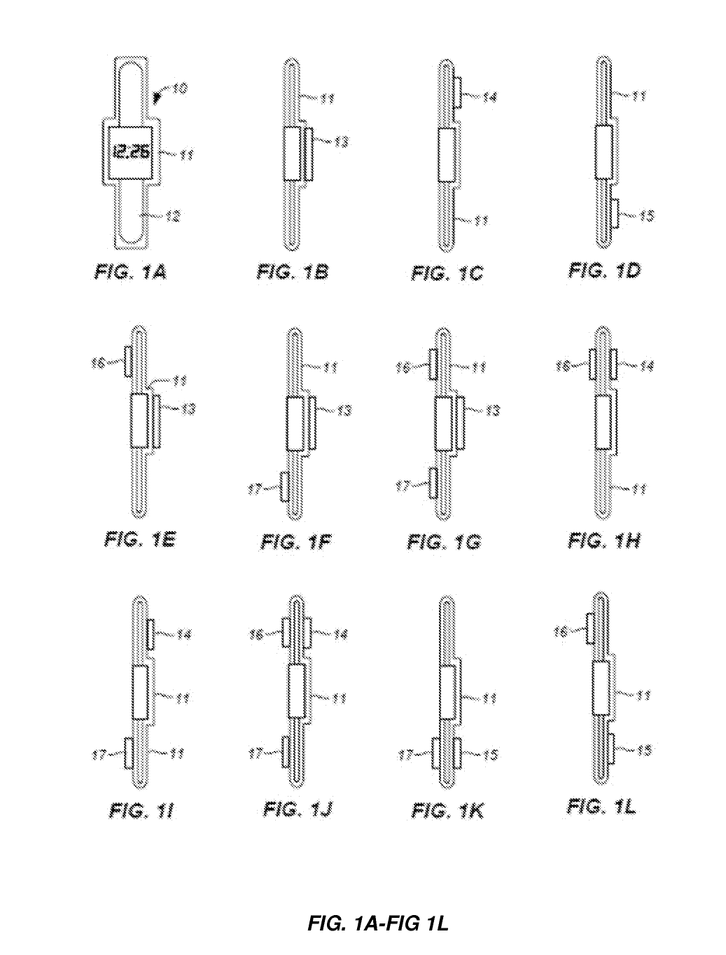

FIGS. 1A through 1L show an embodiment of an attachable monitoring device 10 comprising a cover 11 that fits over a wristwatch 12. The wristwatch 12 may comprise a traditional watch or a smartwatch. A smartwatch may comprise a main body, a display, and two or more straps. A first strap may comprise a camera. One or more of the straps may be replaceable or interchangeable, for example, the second strap. The main body may comprise one or more of a processor, a memory or other storage medium, an input/output system, a wireless transmitter, a speaker, or a microphone. FIG. 1B shows a left lateral view of an embodiment of an attachable monitoring device comprising a cover 11 that fits over a wristwatch, wherein when the cover 11 is coupled with a wristwatch, the sensor 13 is positioned over the posterior surface of the body of the wristwatch so that the sensor 13 is in contact with a skin surface of a user when the wristwatch is worn by a user. FIG. 1C shows an embodiment of an attachable monitoring device comprising a cover 11 that fits over a wristwatch, wherein when the cover 11 is coupled with a wristwatch, the sensor 14 is positioned over a posterior surface of a first watch strap of the wristwatch so that the sensor 14 is in contact with a skin surface of a user when the wristwatch is worn by a user. FIG. 1D shows an embodiment of an attachable monitoring device comprising a cover 11 that fits over a wristwatch, wherein when the cover 11 is coupled with a wristwatch, the sensor 15 is positioned over a posterior surface of a second watch strap of the wristwatch so that the sensor 15 is in contact with a skin surface of a user when the wristwatch is worn by a user. FIG. 1E shows an embodiment of an attachable monitoring device comprising a cover 11 that fits over a wristwatch, wherein when the cover 11 is coupled with a wristwatch, the sensors 13 and 16 are positioned over the posterior surface of the body of the wristwatch and over an anterior surface of a first watch strap of the wristwatch so that the sensor 13 is in contact with a skin surface of a user when the wristwatch is worn by a user and the sensor 16 is may be contacted by a different skin surface of a user when the wristwatch is worn by a user. A different surface may comprise a skin surface of a different extremity than the one on which the wristwatch is worn. For example, a user wearing a wristwatch coupled to the attachable monitoring device may wear the wristwatch coupled to the attachable monitoring device on a left wrist so that sensor 13 contacts a skin surface of the left upper extremity of the user, and the user may contact sensor 16 with a skin surface of either a right upper extremity or left lower extremity. In an embodiment, sensors 13 and 16 comprise electrodes, and a user who wears the attachable monitoring device 10 coupled to a wristwatch measures a lead I ECG when the user contacts sensor 16 with a skin surface of a right upper extremity while sensor 13 is in contact with the user's skin surface on the user's left upper extremity, and measures a lead III ECG when the user contacts sensor 16 with a left lower extremity while sensor 13 is in contact with the user's skin surface on the user's left upper extremity. FIG. 1F shows an embodiment of an attachable monitoring device comprising a cover 11 that fits over a wristwatch, wherein when the cover 11 is coupled with a wristwatch, the sensors 13 and 17 are positioned over the posterior surface of the body of the wristwatch and over an anterior surface of a first watch strap of the wristwatch so that the sensor 13 is in contact with a skin surface of a user when the wristwatch is worn by a user and the sensor 17 is may be contacted by a different skin surface of a user when the wristwatch is worn by a user. FIG. 1G shows an embodiment of an attachable monitoring device comprising a cover 11 that fits over a wristwatch, wherein when the cover 11 is coupled with a wristwatch, the sensors 13, 16, and 17 are positioned over the posterior surface of the body of the wristwatch, over an anterior surface of a first watch strap of the wristwatch, and over an anterior surface of a second watch strap of the wristwatch so that the sensor 13 is in contact with a skin surface of a user when the wristwatch is worn by a user and the sensors 16 and 17 is may be contacted by a different skin surface of a user when the wristwatch is worn by a user. For example, a user wearing a wristwatch coupled to the attachable monitoring device may wear the wristwatch coupled to the attachable monitoring device on a left wrist so that sensor 13 contacts a skin surface of the left upper extremity of the user, and the user may contact sensor 16 with a skin surface of a right upper extremity and contacts sensor 17 with a left lower extremity. In an embodiment, sensors 13, 16, and 17 comprise electrodes, and a user who wears the attachable monitoring device 10 coupled to a wristwatch measures a lead I ECG when the user contacts sensor 16 with a skin surface of a right upper extremity while sensor 13 is in contact with the user's skin surface on the user's left upper extremity, measures a lead II ECG when the user contacts sensor 16 with a skin surface of a right upper extremity while sensor 17 contacts a skin surface of a left lower extremity, and measures a lead III ECG when the user contacts sensor 16 with a left lower extremity while sensor 13 is in contact with the user's skin surface on the user's left upper extremity. FIG. 1H shows an embodiment of an attachable monitoring device comprising a cover 11 that fits over a wristwatch, wherein when the cover 11 is coupled with a wristwatch, the sensor 14 is positioned over a posterior surface of a first watch strap of the wristwatch so that the sensor 14 is in contact with a skin surface of a user when the wristwatch is worn by a user, and the sensor 16 is positioned over the anterior surface of the first watch strap so that the user may contact sensor 16 with a different skin surface than the skin surface in contact with sensor 14.

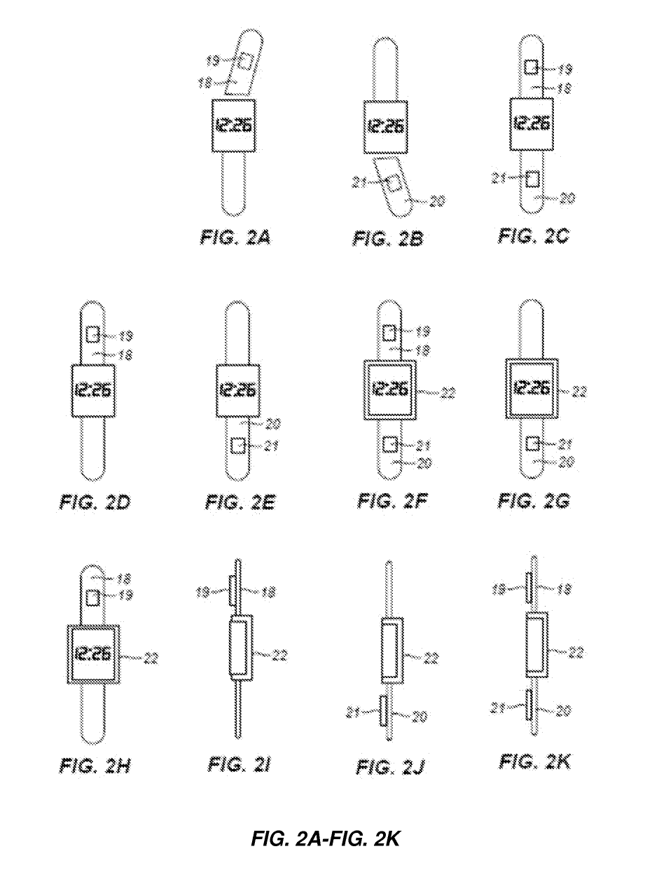

FIGS. 2A-2K show an attachable monitoring device comprising a watchband comprising of straps 18 and 20. In an embodiment, an attachable monitoring device having sensor electrodes incorporated into or over the straps of the device. The wearable monitoring device may comprise a smartwatch. One or more of the straps of the attachable monitoring device may have two or more sensing electrodes incorporated thereon. For example, a first electrode may be placed over a first side of the strap and a second electrode may be placed over a second side of the strap. The first electrode may be contacted with a right or left hand or fingers while the second electrode contacts the opposite wrist to measure heart rate, an electrocardiogram, body fat percentage, hydration, etc. The sensor electrode(s) may be in communication with the processor of the attachable monitoring device through a wired or a wireless connection as described above. In an embodiment, an attachable monitoring device having sensor electrodes snapped onto the device. As shown in FIG. 2C, the sensor accessory may be configured to snap onto the strap of the attachable monitoring device. For example, a first electrode may be placed over a first side of the strap and a second electrode may be placed over a second side of the strap. The first electrode may be contacted with a right or left hand or fingers while the second electrode contacts the opposite wrist to measure heart rate, an electrocardiogram, body fat percentage, hydration, etc. The sensor accessory may be in communication with the processor of the attachable monitoring device through a wired or a wireless connection as described above. In an embodiment, an attachable monitoring device comprises a replaceable strap with sensor electrodes. In an embodiment, an attachable monitoring device may comprise a smartwatch such as the attachable monitoring device. The attachable monitoring device may comprise one or more replaceable straps. A sensor accessory having sensor electrodes may be provided as a replaceable strap for the attachable monitoring device. A first electrode of the accessory may be placed over a first side of the strap and a second electrode of the accessory may be placed over a second side of the strap. The first electrode may be contacted with a right or left hand or fingers while the second electrode contacts the opposite wrist to measure heart rate, an electrocardiogram, body fat percentage, etc. The sensor accessory may be in communication with the processor of the attachable monitoring device through a wired or a wireless connection as described above. Watch straps 18 and 20 comprise sensors incorporated into or positioned over the main body of the device. FIGS. 2A and 2B show anterior views of two embodiments of an attachable monitoring device comprising a watchband comprising two straps 18 and 20, wherein in a first embodiment, shown in FIG. 2A sensor 19 may be incorporated into or positioned over a first strap 18, and, in a second embodiment, shown in FIG. 2B, a sensor 21 may be incorporated into or positioned over a second strap 20. In an embodiment, either or both straps 18 and 20 removably couple with a watch housing, so that, for example, a watch strap 18 or 20 comprising a sensor 19 or 21 may be used with different watches by removing the strap from a first watch and coupling it with a second watch. FIG. 2C shows an anterior view of an embodiment of an attachable monitoring device comprising of a watchband which comprises two straps 18 and 20 which each respectively comprise two sensors 19 and 21. In an alternate embodiment, a first sensor 19 may be positioned on an anterior surface of a watch strap 18 while a second senor 21 is positioned on a posterior surface of a watch strap 20. FIGS. 2F-2K show embodiments of an attachable monitoring device comprising snap-on sensors. FIGS. 2F-2H show anterior views of embodiments wherein sensors 19 and 21 are incorporated in or positioned over watch straps 18 and 20 and a snap-on sensor 22 is coupled with the watch housing. FIG. 2I-2K show left lateral views of embodiments of an attachable monitoring device comprising multiple snap on sensors. FIG. 2I shows a left lateral view of a sensor 19 snap-on coupling with a watchband 18 and a sensor 22 snap-on coupling with a watch housing. FIG. 2K shows sensors 19 and 20 respectively snap-on coupling with watch straps 18 and 20 and sensor 22 snap-on coupling with a watch housing.

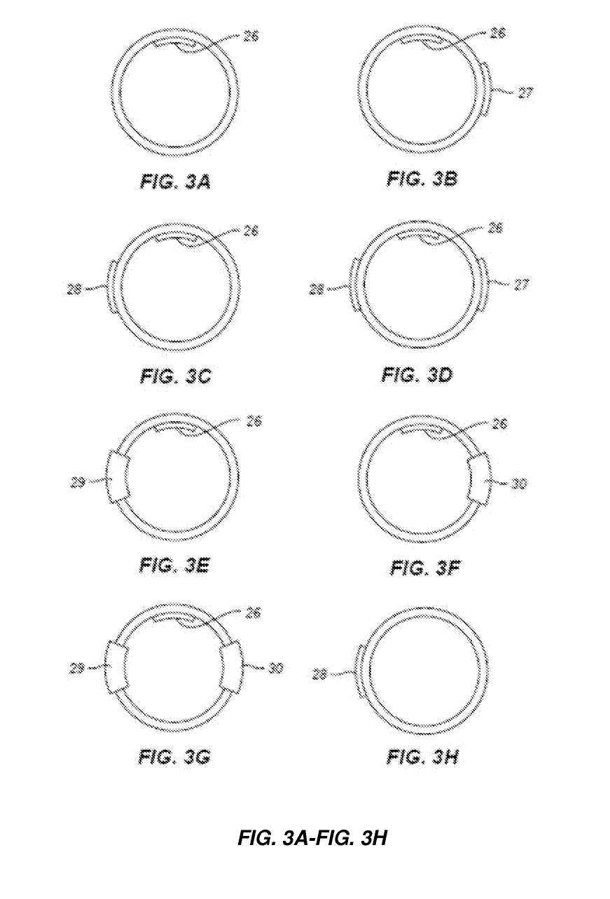

FIGS. 3A-3H show anterior views of an embodiment of an attachable monitoring device comprising an essentially round jewelry such as, for example, a ring, bracelet, or earing. FIGS. 3A-3H show alternate positioning of sensors 26-30, as shown an attachable monitoring device comprising a ring jewelry may have one or more sensors positioned on an inner or outer surface. FIGS. 3E, 3F, and 3G, also show embodiments wherein sensors 29 and 30 snap-on to a ring jewelry and may be thus simultaneously positioned on the inner and outer surfaces of a ring jewelry.

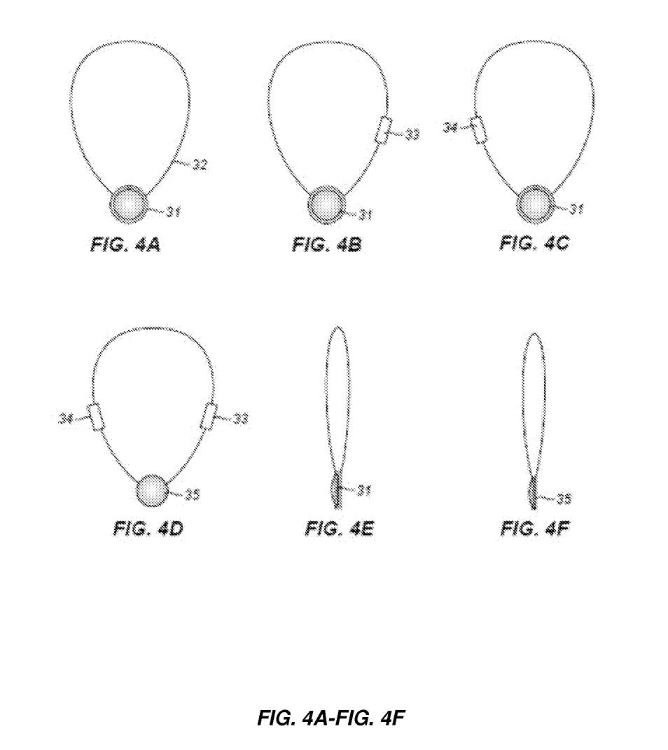

FIGS. 4A-4F show anterior and lateral views of an attachable monitoring device comprising a necklace with a pendant. FIGS. 4B and 4C both show anterior views of embodiments of a snap-on sensor 31 that couples with a pendant and sensors 33 and 34 that are positioned on the necklace chain. FIG. 4E shows a left lateral view of a sensor 31 snap-on coupled with a pendant. FIGS. 4D and 4F show embodiments in which pendant 35 comprises a sensor.

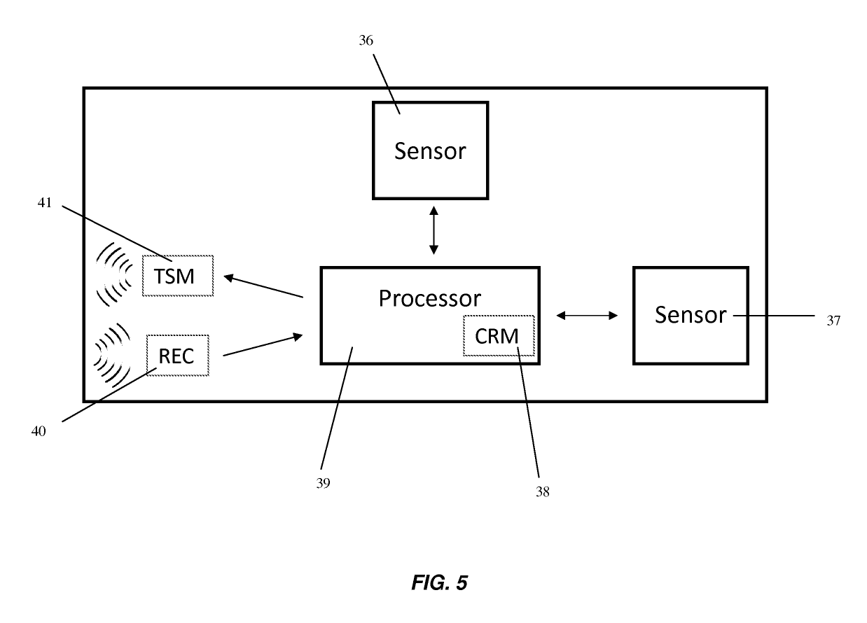

FIG. 5 shows a schematic representation of an embodiment of an attachable monitoring device. An attachable monitoring device may comprise one or more of one or more sensors 36 and 37, a processor 39, a computer readable media 38, a receiver 40, and a transmitter 41. Sensors 36 and 37 are configured to sense biometric parameter data from a user, and to transmit the data to a processor 39. A processor 39 may be configured to process data received from various sensors. For example, in an embodiment, sensors 36 and 37 comprise electrodes that record electric potentials. In the same embodiment, the electric potentials transmitted from sensors 36 and 37 may be transmitted to processor 39 which transforms the sensed data into an electrocardiogram. In an embodiment, processor 39 may further analyze data received from sensors 36 and 37, for example, by comparing the received data to normal data values. In an embodiment, a processor 39 is configured to analyze an electrocardiogram recorded by sensors 36 and 37. For example, a processor 39 may analyze an electrocardiogram recorded from a user for arrhythmias, heart rate variability, or heart rate turbulence. A processor 39 is functionally coupled to a computer readable media 38 which comprises software which comprises executable commands that may be executed by processor 39. For example, software stored on a computer readable media 38 may cause processor 39 to analyze an electrocardiogram. Software stored on a computer readable media 38 may cause a processor to encode biometric parameter data for transmission. Processor 39 is coupled to receiver 40 which is configured to receive a wireless transmission from a computing device. In an embodiment, receiver 40 transmits a received wireless transmission to the processor 39. In the same embodiment, a received wireless transmission comprises an executable command that causes processor 39 to perform some function. For example, a received wireless transmission may cause a processor 39 to activate one or more sensors causing the one or more sensors to sense a biometric parameter from a user. For example, a received wireless transmission may cause a processor 39 to transmit a signal to transmitter 41. A wireless transmitter 41 is coupled to processor 39. In an embodiment, a wireless transmitter 41 is configured to transmit a wireless signal to a computing device. For example, in an embodiment, a transmitter transmits an electrocardiogram recorded with the wearable measuring device to a computing device with a screen display, causing the screen display of the computing device to display the electrocardiogram. In an embodiment, an attachable monitoring device comprises a watchband which comprises a body comprising one or more straps which comprise one or more of sensors 36 and 37, processor 39, computer readable media 38, receiver 40, and transmitter 41. In the same embodiment, a wireless signal is received by receiver 40 from a mobile computing device comprising an executable command that may be carried out by processor 39. In the same embodiment, the executable command causes processor 39 to cause sensors 36 and 37 to sense a biometric parameter of a user and cause transmitter 41 to transmit the biometric parameter sensed by sensors 36 and 37 to a computing device. In an embodiment, a computing device transmits an executable command to an attachable monitoring device in response to biometric parameter data of a user. For example, a computing device comprising, for example, a smartphone may receive biometric parameter data of a user from a sensor that is engaged with the user, wherein the biometric parameter data comprises a heart rate. In an embodiment, the smartphone will analyze the received heart rate and make a determination if there is a need to, for example, record an electrocardiogram of the user. When the smartphone determines that there is a need to record an electrocardiogram of the user based on the heart rate data, it will transmit a signal to an attachable monitoring device that, for example, comprises a watchband causing the watchband to record an electrocardiogram. The watchband may transmit the electrocardiogram to the smartphone for display on the smartphone. In an embodiment, the watchband is couple to a smartwatch housing, and transmits the electrocardiogram to the smartwatch for display on the smartwatch screen.

In an embodiment, the attachable monitoring device is in communication with a portable computing device. The portable computing device may comprise a wearable monitoring device, a smartphone, a tablet computer, a laptop computer, or the like. The portable computing device may be in communication with the attachable monitoring device through a wired (e.g., Lightning, USB, FireWire, or the like) or a wireless connection (e.g., WiFi, cellular, Bluetooth classic, Bluetooth low energy, NFC, ultrasound communication, or the like) through the wireless transmitter and/or speaker and microphone of the attachable monitoring device. The portable computing device may provide a variety of computing and application functions while the attachable monitoring device may provide a subset of such functions in a more convenient manner (e.g., notifications, time, date, weather, alerts, device control, etc.). An application may be provided for smartphones, tablet computers, wearable monitoring devices, and the like to provide the user with the ability to track various parameters for health. The user may be able to enter into the application(s) his or her weight and diet, for example. In another example, an on-board accelerometer of the device may track the sleep and/or activity levels of the user. Heart rate may be monitored using the on-board camera and flash of the device. In some embodiments, the devices may include on-board electrodes for monitoring heart rate. Accessories for these devices may be used to track activity levels and other health parameters as well.

In an embodiment, an attachable monitoring device may comprise an accelerometer which may be used to detect the activity level and patterns of the user. The attachable monitoring device may be in continuous contact with the user throughout an extended period (e.g., an hour, a half-day, a day, or more) and various physiological parameters may be continuously measured. For example, physiological parameters such as activity level, heart rate, hydration, ECG, etc. may be continuously measured and correlated with one or more of each other. For example, correlating the activity level of the user with the heart rate of the user may provide an indication as to the fitness level of the user. Significant increases in heart rate with trivial activity and/or slow decrease in heart rate after trivial activity may indicate to the user that he or she has a low fitness level and low cardiovascular health. Conversely, insignificant increases in heart rate with substantial activity and/or a healthy decrease in heart rate after significant activity may indicate to the user that he or she has a healthy or high fitness level and a healthy or high cardiovascular health. ECG may also be correlated with activity level to detect any cardiac abnormalities such as arrhythmias that occurs during physical activity. For example, the user may be prompted or notified to take an ECG immediately after a high level of user physical activity as indicated by heart rate, skin hydration, and/or the accelerometer.

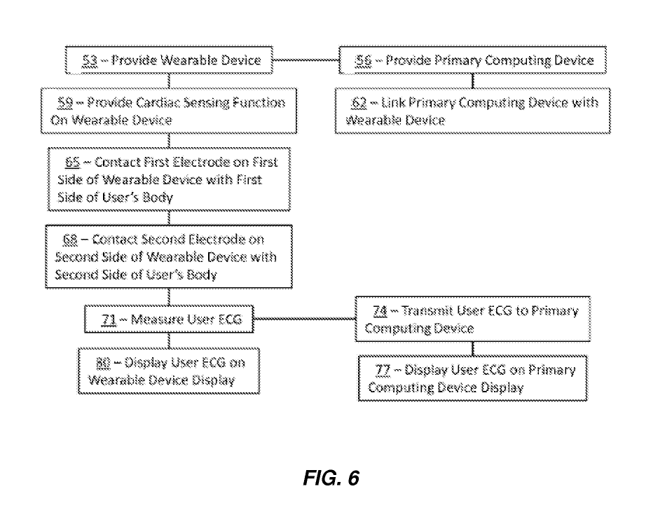

FIG. 6 shows a schematic of a method of measuring an electrocardiogram. The method may comprise a step 53 of providing a wearable computing or other electronic device and a step 59 of providing a cardiac sensing function on the wearable device. In many embodiments, the method 50 may further comprise a step 56 of providing a primary computing device. In a step 62, the primary computing device may be linked with the wearable device.

The method may further include a step 65 of contacting a first electrode on a first side of the wearable device with a first side of a user's body. And, in a step 68, a second electrode on a second side of the wearable device may be contacted with a second side of the user's body. In a step 71, the user's ECG may be measured 71. In a step 80, the user ECG may be displayed on a display of the wearable device. In some embodiments, the user ECG may be transmitted to the primary computing device in a step 74. In a step 77, the user's ECG may be displayed on a display of the primary computing device.

Although the above steps show method 50 of measuring an ECG in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial to the treatment.

One or more of the steps 50 of measuring an ECG may be performed with circuitry (i.e., electrodes, processors, storage elements, etc.) described herein, for example, or one or more of the processor of the sensor accessory, attachable monitoring device, and/or primary computing device or other logic circuitry such as a programmable array logic, a field programmable gate array, or an application specific integrated circuit. The circuitry may be programmed to provide one or more of the steps of the method 50, and the program may comprise program instructions stored on a computer readable memory or programmed steps of the logic circuitry.

As discussed above, a smartwatch or electronic wrist band may comprise a secondary portable computing device of a mobile computing system which includes a primary portable computing device such as a laptop computer, a tablet computer, a smartphone, or the like. The smartwatch may be used to notify the user to take one or more physiological parameter measurements using the mobile computing system.

The mobile computing system may notify the user through the smartwatch or electronic wrist band to take daily ECG measurements at predetermined times. Alternatively or in combination, the mobile computing system may comprise a set of instructions which when executed causes the mobile computing system to monitor the user and determine whether the user should be notified to take a physiological parameter measurement. Such notification can be provided for a variety of reasons. The smartwatch may prompt the user to measure his or her ECG when the user's activity level (as measured continuously by the smartwatch accelerometer, for example) meets a threshold activity level, when the user's heart rate (as measured continuously by the smartwatch heart rate sensor, for example) meets a threshold heart rate, when a heart rate variability (HRV) or a user (as measured and determined continuously by the smartwatch heart rate sensor and an application provided on the mobile computing system, for example) meets a threshold HRV, when a measured blood pressure of the user (as measured by a third device, for example) is met, and/or based on other learned environmental cues. For example, if atrial fibrillation is detected when one or more other physiological parameters are measured to be at a first level, the user may be prompted to take his or her ECG when the same physiological parameters again meet the measured first level. These parameters may include one or more of activity, time of day, location of user or device(s), phone state (charging, unplugged, screen on/off, Wi-Fi on/off, Bluetooth on/off, etc.), skin hydration or sweat levels, blood pressure, or other user input metrics (e.g., user scheduling events). The threshold activity level, heart rate, HRV, blood pressure, and the like may be determined by the user (and input into the mobile computing system) or may be determined based on how well the user fits with a set of predetermined criteria, based on prior physiological measurements using the mobile computing system. The notification may be provided as a vibration of the smartwatch or smartphone and/or a displayed prompt on the display of the smartwatch or electronic wrist band.

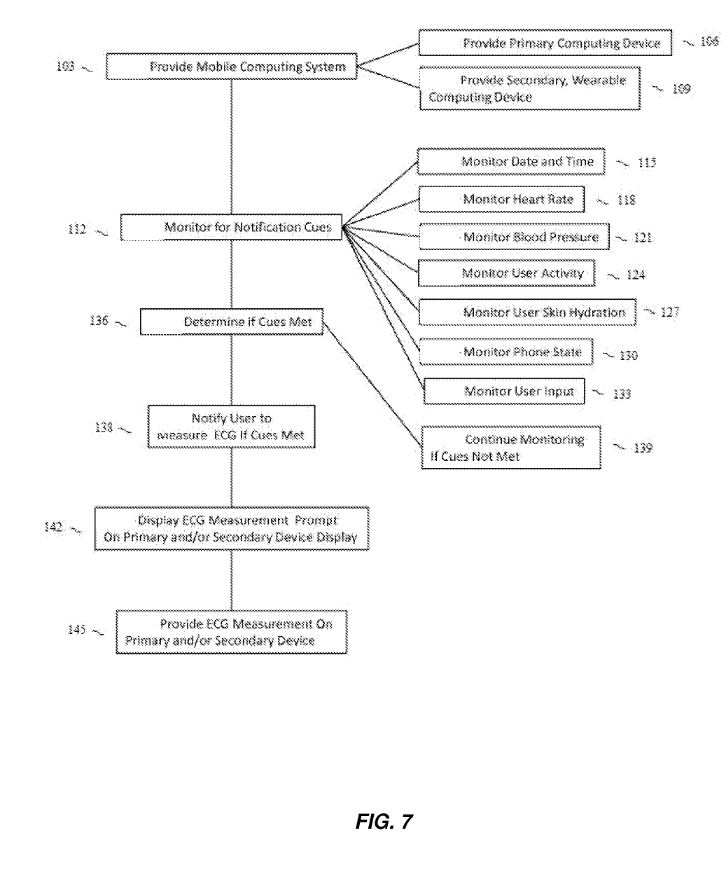

FIG. 7 shows a schematic of method of notifying a user and measuring an ECG of the user. The method may include a step 103 of providing a mobile computing system. For example, a primary computing device may be provided in a step 106 and/or a secondary, wearable monitoring device may be provided in a step 109. The method 100 may include monitoring for notification cues in a step 112. For example, such monitoring may include monitoring date and time in a step 115, monitoring heart rate in a step 118, monitoring blood pressure in a step 121, monitoring user activity in a step 121, monitoring user skin hydration in a step 127, monitoring phone state in a step 130, monitoring user input in a step 133, and/or continuing the monitoring if the cues not met in a step 139.

The method may further comprise determining if cues are met in a step 136. If the cues are met, the method may include notifying the user to measure ECG in a step 138. The method 100 may include a step 122 of displaying an ECG measurement prompt on a display (or displays) of the primary device and/or the secondary device. The ECG measurement may be provided on the primary device and/or the secondary device in a step 115.

Although the above steps show method of measuring an ECG in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial to the treatment.

One or more of the steps of measuring an ECG may be performed with circuitry (i.e., electrodes, processors, storage elements, etc.) described herein, for example, or one or more of the processor of the sensor accessory, wearable monitoring device, and/or primary computing device or other logic circuitry such as a programmable array logic, a field programmable gate array, or an application specific integrated circuit. The circuitry may be programmed to provide one or more of the steps of the method, and the program may comprise program instructions stored on a computer readable memory or programmed steps of the logic circuitry.

As discussed above, a smartwatch or electronic wrist band may comprise a secondary portable computing device of a mobile computing system which includes a primary portable computing device such as a laptop computer, a tablet computer, a smartphone, or the like. The smartwatch may be used to notify the user to take one or more physiological parameter measurements using the mobile computing system.

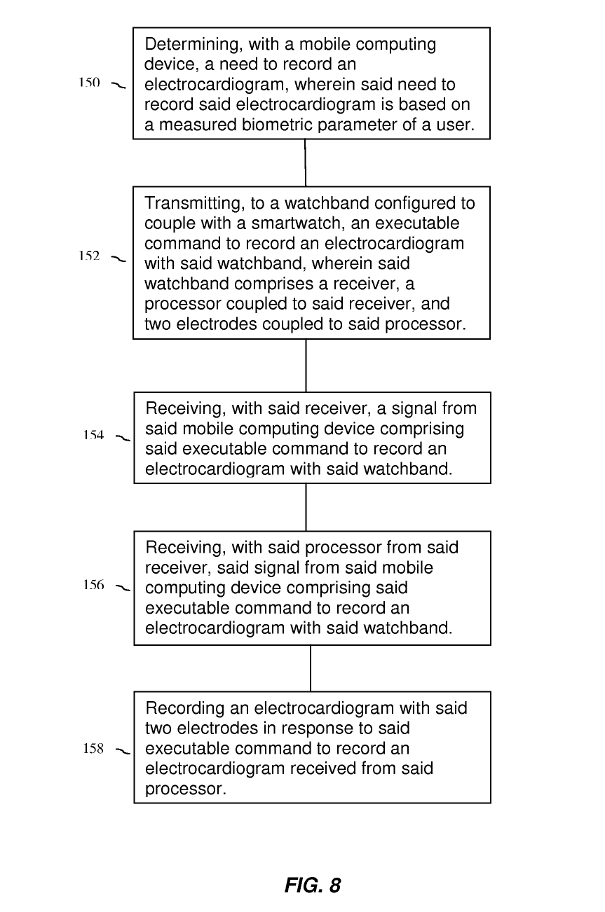

FIG. 8 shows a schematic representation of a method for monitoring a user. In a step 150, a computing device determines whether there is a need to record an electrocardiogram of a user. The need to record the electrocardiogram of the user may be, for example, based on a measured biometric parameter of a user that is sensed by a sensor that is coupled to the computing device.

In a step 152, a wireless signal is transmitted to o a watchband configured to couple with a smartwatch. The wireless signal may comprise an executable command to record an electrocardiogram with said watchband. The watchband comprises a receiver, a processor coupled to said receiver, and two electrodes coupled to said processor.

In a step 154, the watchband receiver receives the signal from the mobile computing device comprising the executable command to record an electrocardiogram with said watchband.

In a step 156, the watchband processor receives the signal from the receiver.

In a step 158, the watchband processor causes the two electrodes to record an electrocardiogram.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for monitoring a biometric parameter of a user, comprising
receiving, by said user, an attachable monitoring device comprising a processor and a sensor, wherein said attachable monitoring device is configured to couple with a wearable monitoring device of said user;
coupling, by said user, said attachable monitoring device with said wearable monitoring device;
sensing, with said sensor, a biometric parameter of said user; and
transmitting said biometric parameter of said user to said wearable monitoring device of said user.

2. The method of claim 1, wherein said coupling step comprises both a physical and functional coupling of said attachable monitoring device with said wearable monitoring device.

3. The method of claim 2, wherein said physical coupling comprises a snap-on coupling.

4. The method of claim 2, wherein said functional coupling comprises a hardwire electronic coupling between said attachable monitoring device and said wearable monitoring device.

5. The method of claim 2, wherein said attachable monitoring device further comprises a wireless transmitter, and said functional coupling comprises a wireless coupling between said attachable monitoring device and said wearable monitoring device.

6. The method of claim 2, wherein said attachable monitoring device comprises a watch band, and wherein said wearable monitoring device comprises a smartwatch.

7. The method of claim 1, wherein said biometric parameter comprises an electrocardiogram.

8. The method of claim 1, wherein said processor is configured to activate said sensor.

9. The method of claim 1, wherein said processor is configured to analyze said sensed biometric parameter.

10. The method of claim 1, further comprising displaying said biometric parameter on a display of said wearable monitoring device.

11. An attachable monitoring device comprising a body having a first surface and a second surface, wherein said body is configured to removably couple with a wearable monitoring device, and wherein said body comprises:
a) a first sensor positioned on said first surface, wherein said first sensor is configured to sense a biometric parameter of a user;
b) a processor coupled to said first sensor; and
c) a coupler configured to couple said body with said wearable monitoring device.

12. The attachable monitoring device of claim 11, wherein said coupler both physically and functionally couples said body with said wearable monitoring device.

13. The attachable monitoring device of claim 12, wherein said physical coupling comprises a snap-on coupling.

14. The attachable monitoring device of claim 12, wherein said functional coupling comprises a hardwire electronic coupling between said attachable monitoring device and said wearable monitoring device.

15. The attachable monitoring device of claim 12, wherein said body further comprises a wireless transmitter, and said functional coupling comprises a wireless coupling between said attachable monitoring device and said wearable monitoring device.

16. The attachable monitoring device of claim 11, wherein said first sensor is configured to measure an electric potential on a first skin surface of a user, and wherein said body further comprises a second sensor positioned on said second surface, wherein said second sensor is coupled to said processor, and wherein said second sensor is configured to measure an electric potential on a second skin surface of said user;

17. The attachable monitoring device of claim 16, further comprising a non-transitory computer-readable storage media encoded with a computer program including instructions executable by said processor to cause said processor to generate an electrocardiogram comprising said electric potential on said first skin surface of said user and said electric potential on said second skin surface of said user.

18. The attachable monitoring device of claim 17, wherein said computer program further causes said processor to transmit said electrocardiogram to said wearable monitoring device.

19. The attachable monitoring device of claim 17, wherein said computer program further causes said processor to instruct said user to contact said first and said second sensors.

20. The attachable monitoring device of claim 11, wherein said wearable monitoring device comprises a smartwatch and said body comprises a watchband.
